(19) United States
(12) Patent Application Publication
(10) Pub. No.: US 2020/0118656 A1
(43) Pub. Date: Apr. 16, 2020

(54) SYSTEMS FOR VALIDATING HEALTHCARE TRANSACTIONS

(71) Applicant: HEALTHGATES INC., Wilmington, DE (US)

(72) Inventors: Francis Arena, Manhasset, NY (US); Matthew Markham, San Francisco, CA (US); William Newman, New York, NY (US)

(21) Appl. No.: 16/654,186

(22) Filed: Oct. 16, 2019

Related U.S. Application Data
(60) Provisional application No. 62/746,310, filed on Oct. 16, 2018.

(51) Int. Cl. G16H 10/60; G06Q 20/14; G06Q 20/40; G16H 15/00

(52) U.S. Cl. CPC G16H 10/60; G16H 15/00; G06Q 20/401; G06Q 20/14

(57) ABSTRACT

A method for validating healthcare transactions via a web-based platform includes: obtaining a preliminary diagnosis from a remote computing device; comparing the preliminary diagnosis to a predetermined diagnosis criteria; determining whether the preliminary diagnosis satisfies the predetermined diagnosis criteria; authorizing payment to a predetermined entity when the preliminary diagnosis is determined to satisfy the predetermined diagnosis; generating a unique token when payment is authorized; recording a transaction in a distributed ledger based on the unique token to establish a recorded transaction; associating the unique token with the recorded transaction; obtaining a fulfillment request from the remote computing device; validating the recorded transaction based on the unique token to establish a validation; and transmitting an indication to fulfill the fulfillment request based on the validation to the remote computing device.

### TECHNICAL FIELD

The present disclosure relates to healthcare transactions and, more particularly, to systems for validating healthcare transactions.

### BACKGROUND

The delivery of targeted medical therapies in a heavily regulated environment has become a difficult and taxing adventure for physicians, insurance companies, and patients. Antiquated and inefficient protocols and procedures make the difficult work of properly authorizing such therapies even more difficult. Many doctors still need to use fax machines, as if it were 1980 and not nearly 2020. Even with computers, many physicians, insurance companies, and specialty pharmacies still experience unnecessary delays because of low-quality systems. Authorization delays can cost not only time and money to the physician and hospital practice, but also jeopardize the lives of patients.

One of the primary sources of climbing costs is the health insurance prior authorization (PA) process. With 30% of Medicare charges done in error, this area should not be ignored. Insurers, physicians, patients, and the pharmaceutical industry are disconnected from each other. While inefficiencies are inherent in any supply chain, and within all industries, the gaps between each party in the healthcare system pose potentially deadly problems for patients. Thus, one of the primary flaws in the current system is the use of outdated, and often tedious, treatment authorization protocols. The reason electronic PA systems have not been widely adopted is because each health insurer has its own set of PA requirements, and they have not agreed on one electronic system. Accordingly, there is continued interest in the PA process.

### SUMMARY

In accordance with aspects of the disclosure, a method for validating healthcare transactions via a web-based platform is presented. The method includes: obtaining, by a server, a preliminary diagnosis from a remote computing device; comparing, by the server, the preliminary diagnosis to a predetermined diagnosis criteria; determining, by the server, whether the preliminary diagnosis satisfies the predetermined diagnosis criteria; authorizing payment, by the server, to a predetermined entity when the preliminary diagnosis is determined to satisfy the predetermined diagnosis; generating a unique token, by the server, when payment is authorized; recording a transaction, by the server, in a distributed ledger based on the unique token to establish a recorded transaction; associating, by the server, the unique token with the recorded transaction; obtaining, by the server, a fulfillment request from the remote computing device; validating, by the server, the recorded transaction based on the unique token to establish a validation; and transmitting, by the server, an indication to fulfill the fulfillment request based on the validation to the remote computing device.

In an aspect of the present disclosure, the transaction may include an authorization for the payment to the predetermined entity and the preliminary diagnosis.

In another aspect of the present disclosure, the preliminary diagnosis may include a preliminary diagnosis information.

In yet another aspect of the present disclosure, the preliminary diagnosis may include a prescription information.

In a further aspect of the present disclosure, the prescription information may include a name of a prescribing physician, a nature of a physician's practice, a name prescribed prescription, a dosage of the name prescribed prescription, a unique transaction serial number, and/or a transaction timestamp.

In yet a further aspect of the present disclosure, the unique token may include data related to the preliminary diagnosis.

In an aspect of the present disclosure, the unique token may further include a unique identifier.

In another aspect of the present disclosure, the preliminary diagnosis may include diagnosis information, scan data captured by medical imaging systems, and/or sample test data.

In yet another aspect of the present disclosure, the method may further include obtaining, by the server, an indication that the fulfillment request has been fulfilled and displaying an alert, on a user device, that the fulfillment request has been fulfilled.

In accordance with aspects of the disclosure, a system for validating healthcare transactions via a web-based platform is presented. The system includes a server comprising: a processor and a memory storing instructions. The instructions when executed by the processor, cause the system to: obtain a preliminary diagnosis from a remote computing device; compare the preliminary diagnosis to a predetermined diagnosis criteria; determine whether the preliminary diagnosis satisfies the predetermined diagnosis criteria; authorize payment to a predetermined entity when the preliminary diagnosis is determined to satisfy the predetermined diagnosis; generate a unique token when payment is authorized; record a transaction in a distributed ledger based on the unique token to establish a recorded transaction; associate the unique token with the recorded transaction; obtain a fulfillment request from the remote computing device; validate the recorded transaction based on the unique token to establish a validation; and transmit an indication to fulfill the fulfillment request based on the validation to the remote computing device.

In an aspect of the present disclosure, the transaction may include an authorization for the payment to the predetermined entity and the preliminary diagnosis.

In another aspect of the present disclosure, the preliminary diagnosis may include a preliminary diagnosis information.

In yet another aspect of the present disclosure, the preliminary diagnosis may include a prescription information.

In a further aspect of the present disclosure, the prescription information may include a name of a prescribing physician, a nature of a physician's practice, a name prescribed prescription, a dosage of the name prescribed prescription, a unique transaction serial number, and/or a transaction timestamp.

In yet a further aspect of the present disclosure, the unique token may include data related to the preliminary diagnosis.

In an aspect of the present disclosure, the unique token may further include a unique identifier.

In another aspect of the present disclosure, the preliminary diagnosis may include diagnosis information, scan data captured by medical imaging systems, and/or sample test data.

In yet another aspect of the present disclosure, the instructions when executed further cause the system to obtain an indication that the fulfillment request has been fulfilled, and display an alert, on a user device, that the fulfillment request has been fulfilled.

In another aspect of the present disclosure, the instructions when executed, further cause the system to transmit the unique token to a user device.

In accordance with aspects of the disclosure, a non-transitory computer-readable medium storing a program for validating healthcare transactions via a web-based platform is presented. The program includes instructions which, when executed, cause a server to: obtain a preliminary diagnosis from a remote computing device; compare the preliminary diagnosis to a predetermined diagnosis criteria; determine whether the preliminary diagnosis satisfies the predetermined diagnosis criteria; authorize payment to a predetermined entity when the preliminary diagnosis is determined to satisfy the predetermined diagnosis; generate a unique token when payment is authorized; record a transaction in a distributed ledger based on the unique token to establish a recorded transaction; associate the unique token with the recorded transaction; obtain a fulfillment request from the remote computing device; validate the recorded transaction based on the unique token to establish a validation; and transmit an indication to fulfill the fulfillment request based on the validation to the remote computing device.

Further details and aspects of various embodiments of the disclosure are described in more detail below with reference to the appended figures.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure. The figures depict various embodiments of the present disclosure for purposes of illustration only. One skilled in the art will readily recognize from the following discussion that alternative embodiments of the structures and methods illustrated herein may be employed without departing from the principles of the present disclosure described herein.

FIG. 1 is a schematic view of a system for validating healthcare transactions in accordance with the principles of the present disclosure;

FIG. 2 is a schematic view of a method for validating healthcare transactions via the system of FIG. 1; and

FIG. 3 is a block diagram of components associated with a workstation or workstations configured for use with the system of FIG. 1.

### DETAILED DESCRIPTION

Embodiments of the present disclosure are described in detail with reference to the drawings, in which like reference numerals designate identical or corresponding elements in each of the several views.

The present disclosure is directed to a blockchain-based solution that provides the current system's benefits, but faster, more accurately, and with the potential to save millions of dollars. In particular, the present disclosure provides a seamless, immediate, and accurate transmittal of patient data via a blockchain protocol, which allows for the nearly instantaneous authorization of targeted medical therapies to patients. It is designed to function as an integrated system that works across insurance companies, allowing for use by all participants in the healthcare system. It accomplishes this by electronically approving targeted or regulated therapies while the patient is still in the physician's office or clinic (without ever touching a fax machine). This will eliminate the time lag for approval or denial of certain therapies. It will also eliminate the need for peer-to-peer reviews of patient data. It will also enable specialty pharmacies to deliver medications faster than they do now.

In short, the present disclosure enables physicians, insurance companies, patients, and specialty pharmacies to deliver the proper therapies in a swift and streamlined manner. In particular, its focus is on the new era of targeted or “personalized” medicines that have stampeded into the medical scene in such specialties as oncology, hematology, rheumatology, gastroenterology, and neurology.

The present disclosure is directed to a blockchain-based system for the transmission of patient data. It is designed to improve the speed and efficiency of the communication between the pharmaceutical industry, health care providers, insurance industry, and patients. It does not just “eradicate the fax machine.” It replaces a broken PA system that often significantly delays critical treatment. It replaces distant bureaucrats with the immediate expertise of cutting-edge experts. And it seeks to improve upon a system where 50% of initial pre-authorizations are denied.

By confirming that the correct treatments are provided to the correct patients, the system reduces the role of human error, and it provides security to insurance companies that costly therapies are being ordered against published guidelines. By reducing bureaucracy, it gets life-saving treatments to patients faster.

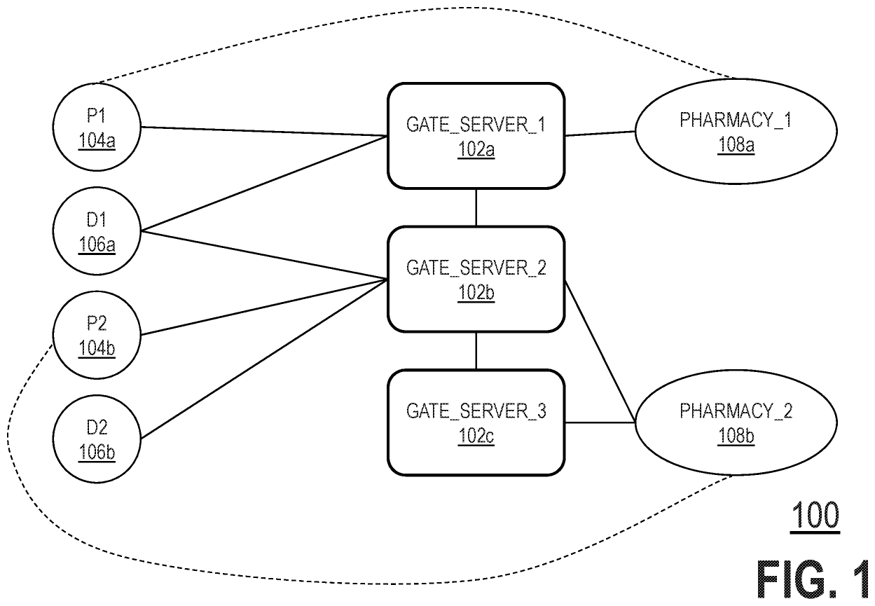

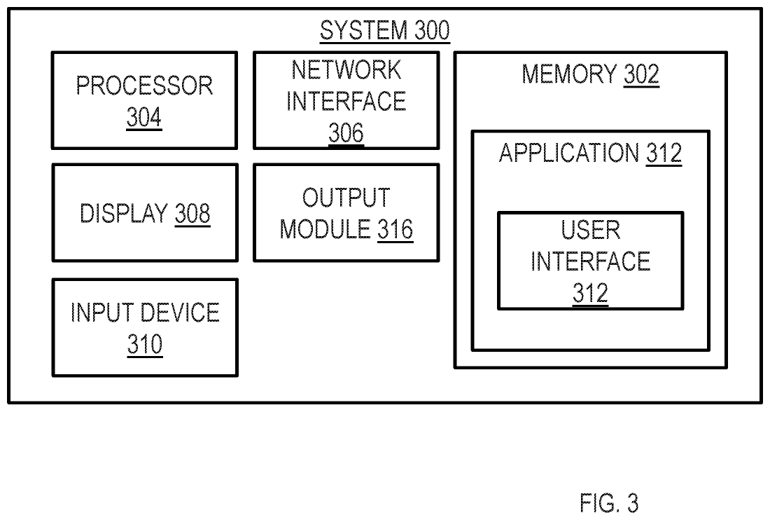

Referring now to FIG. 1, illustrated is a system for validating healthcare transactions, the system designated generally 100. The system includes healthcare transaction servers 102a-102c, patient workstations 104a, 104b, clinician workstations 106a, 106b, and pharmacy workstations 108a, 108b. Depending on the configuration of the network, any or all of the components illustrated may be configured to communicate via a network interface (not explicitly shown; see FIG. 3, network interface 306). For example, data signals may be transmitted between the various components of the system in order to transmit and validate the approval of a pharmaceutical transaction (e.g., the approval to disperse a drug either prescribed by a clinician or purchased over the counter). This communication may be achieved by transmitting the various data signals either via wired, wireless, or a combination of wired and wireless connections. While FIG. 1 illustrates an embodiment in which communication between patient workstations 104a, 104b with pharmacy workstations 108a, 108b may be direct, and clinician workstations 106a, 106b are operably connected to the pharmacy workstations 108a, 108b, it will be understood that any of the illustrated workstations may be operably or directly in communication with one another.

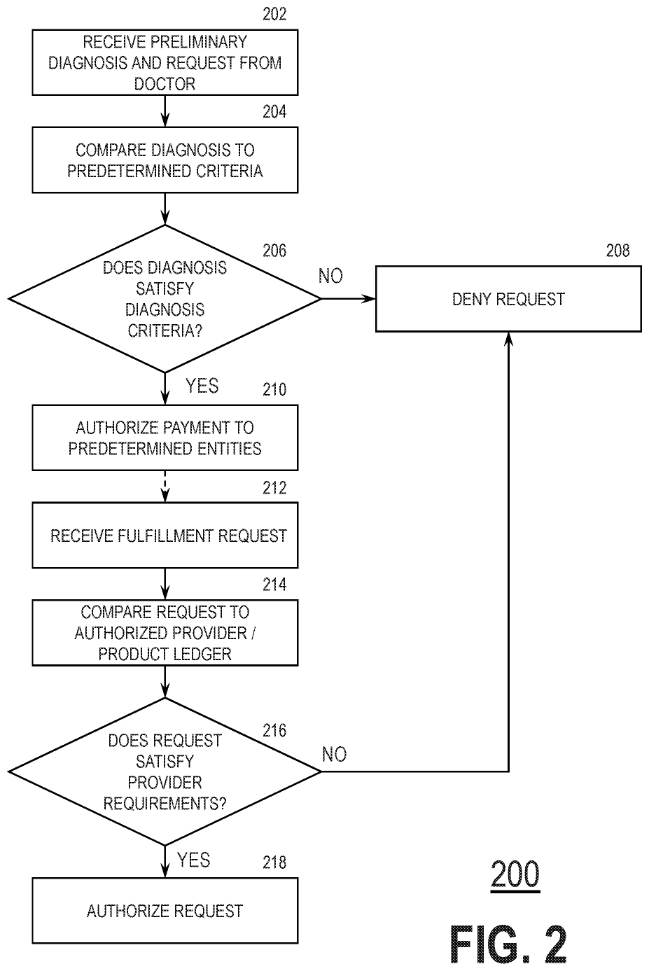

Referring now to FIG. 2, illustrated is a method for validating healthcare transactions via the system of FIG. 1, the method referred to generally as process 200. Initially, once a clinician has assessed a state of health of a patient, the clinician or clinician staff may enter prescription data into a clinician workstation 106a, 106b (block 202). The prescription data may include information such as a chemical compound to be given to a patient, the dosage to be administered (e.g., amount and frequency at which the chemical compound should be administered). In addition to the prescription information, the clinician or clinician staff may enter diagnosis information (e.g., that a patient has epithelial carcinoma of the ovary, fallopian tube cancer, primary peritoneal carcinomatosis, etc.), scan data captured by medical imaging systems (e.g., CT imaging data), sample test data (e.g., biopsy results, blood test results, etc.) and the like. Once input by the clinician or clinician staff at the clinician workstation 106a, 106b, the clinician workstation 106a, 106b transmits the input prescription information and diagnosis information to one or more of the healthcare transaction servers 102a-102c. Once received by the healthcare transaction servers 102a, 102c, the prescription information, and diagnosis information is transmitted to the remaining connected healthcare transaction servers 102a, 102c. Each healthcare transaction server 102a-102c may continue to analyze the prescription information and diagnosis information to determine whether to authorize or deny the prescription delivery request (blocks 204-218). The healthcare transaction server 102a-102c may maintain a ledger (e.g., Etherium) which records each transaction, including information received from the clinician workstation 106a, 106b, the patient workstation 104a, 104b, and/or the pharmacy workstation 108a, 108b. Additionally, or alternatively, the healthcare transaction server 102a-102c may generate one or more packets of information or tokens for approved transactions, which may later be compared when determining whether a transaction is approved, as will be discussed below in greater detail.

Ethereum is an open source, public, blockchain-based distributed computing platform and operating system featuring smart contract (scripting) functionality. Ethereum provides a decentralized virtual machine, the Ethereum Virtual Machine (EVM), which can execute scripts using an international network of public nodes. The virtual machine's instruction set, in contrast to others like Bitcoin Script, is thought to be Turing-complete. The Ethereum Virtual Machine (EVM) is the runtime environment for smart contracts in Ethereum. It is a 256-bit register stack, designed to run the same code exactly as intended. It is the fundamental consensus mechanism for Ethereum.

The diagnosis and/or the prescription data may be compared to a set of predetermined criteria (block 204) and, based on the comparison, the diagnosis and/or prescription data may be approved (“YES” at block 206) or disapproved (“NO” at block 206). For example, if the prescription data is received indicating that a patient is prescribed a chemical compound intended for long-term treatment (e.g., to treat a degenerative condition, disease which is not expected to have a finite duration, etc.) but the treatment is outside of a normal dosing sequence for the particular condition being treated (e.g., the chemical compound proscribed is typically administered after an initial chemical compound is administered), the chemical compound may be identified as an inappropriate or mismatched compound for treatment of the diagnosed condition. In the case of recurring prescriptions, process 200 may be reiterated by repeating the comparison of the diagnosis to the prescription data and, compared to the predetermined criteria based on the time between the last authorization (block 218) and the current requested authorization.

Once the comparison is performed, if it is determined that the diagnosis and prescribed chemical compound match the predetermined criteria (“YES” at block 206), process 200 continues to block 210. Otherwise, if it is determined that the diagnosis and the prescribed chemical compound are mismatched, (“NO” at block 206) process 200 continues to block 208, and the request is denied. Upon multiple iterations (e.g., when verifying multiple prescriptions at multiple points in time), the transaction servers 102a-102c may receive additional information from the clinician workstation 106a, 106b to determine if the prescription is still valid. For example, the transaction servers 102a-102c may receive genotype information and based on the genotype information determine whether certain mutations are present or absent, and by extension whether the prescribed chemical compound is an appropriate form of treatment. In a similar manner, the transaction servers 102a-102c may confirm a patient's suitability for generic therapy, a patient's suitable method of administration (e.g., oral, intravenous) as well as a suitable dispersal period (e.g., once a day, once every other day). Additional pathology samples may be received and, based on the samples, the resistance (or lack thereof) of the chemical compound prescribed may be assessed to determine whether the chemical compound is still effective (e.g., if a certain amount of cells are present indicative of a disease, etc.).

When a diagnosis is received from a clinician workstation 106a, 106b, the diagnosis may be analyzed to determine if the diagnosis is appropriate. For example, if a chemical compound is prescribed for the treatment of Chron's disease, but the patient is prescribed the chemical compound without a test confirming the presence of the disease in the patient, the diagnosis may be identified as faulty. Over time, the predetermined criteria by which the patient's diagnosis is compared to may be updated as is necessary in view of advances in medical research. In embodiments, the healthcare transaction servers 102a-102c may transmit one or more queries when comparing the diagnosis to the predetermined criteria to the patient workstation 104a, 104b, and/or the clinician workstation 106a, 106b to determine if the diagnosis and request are appropriate. For example, the healthcare transaction servers 102a-102c may receive a request from a clinician workstation 160a, 106b, to distribute a pain relief prescription to a patient. The healthcare transaction servers 102a-102c may compare the request to a preexisting patient history and determine if the pain relief prescription is appropriate during the comparison (block 204). When the healthcare transaction servers 102a-102c determines that the diagnosis is compatible with the patient's health history (e.g., the pain relief prescription was prescribed in response to the patient receiving a dental implant), the healthcare transaction servers 102a-102c may authorize the request (block 210). Alternatively, if the pain relief medication is determined to be prescribed for an incompatible diagnosis (e.g., treatment of a minor cavity), the healthcare transaction servers 102a-102c may deny the request (block 208). The healthcare transaction servers 102a-102c may also transmit one or more follow-up queries for the clinician to the clinician workstation 106a, 106b and, based on responses received by the healthcare transaction servers 102a-102c from the clinician workstation 106a, 160b, suggest and/or authorize payment for an appropriate prescription.

In response to determining the diagnosis satisfies the diagnosis criteria (block 206), the healthcare transaction servers 102a-102c marks the transaction as approved and authorizes payment for the purchase of the prescribed chemical compound (block 210). Specifically, the healthcare transaction servers 102a-102c may populate an index of prescriptions which may be filled (e.g., to disperse ten doses of a chemical compound every ten days) which tracks a treatment plan, and store the index in an entry on the ledger (e.g., a blockchain based distributed ledger), the entry associated with the clinician and the patient. If the authorization is ultimately approved for dispersal to the patient (“YES” at block 216), then the entry in the ledger may include a flag indicating that the chemical compound was approved to be delivered to the patient. In embodiments, the data including the authorized prescription may be transmitted as a package of data or a token generated by the healthcare transaction servers 102a-102c, the token (upon generation) being associated with the entry in the ledger. The token, upon generation, may be transmitted to the patient workstation 104a, 104b, and/or to the pharmacy workstation 108a, 108b. The token may include relevant prescription data stored in the entry of the ledger such as, without limitation, the name of the prescribing physician, the nature of the physician's practice, the name and dosage of the prescribed prescription, and unique transaction information such as a transaction serial number, a transaction timestamp, etc. The token may include, for example, an 18 decimal ERC20 compliant token. The token may be stored in a ERC20 compatible wallet.

The healthcare transaction servers 102a-102c may receive a fulfillment request from a remote computing device (block 212). For example, when a patient requests a prescribed prescription, the healthcare transaction servers 102a-102c may receive transaction information from a pharmacy workstation 108a, 108b, indicating that the patient is requesting the prescription. The transaction information may include, without limitation, the name of the prescribed medication, the dosage prescribed, and an expiration date for the prescription. In embodiments, the patient workstation 104a, 104b, and/or the clinician workstation 106a, 106b may transmit the token generated when the prescription was authorized to the healthcare transaction servers 102a-102c.

The healthcare transaction servers 102a-102c may then compare the transaction information and/or the token to the authorized request stored in the entry of the ledger (block 214). For example, the healthcare transaction server 102a-102c may compare a prescribed prescription with a predetermined set of approved prescriptions associated with an insurer that is insuring the patient. When the healthcare transaction server 102a-102c determines the request satisfies predetermined requirements for fulfillment (“YES” at block 216), the healthcare transaction server 102a-102c transmits an indication to fill the request to the pharmacy workstation 108a, 108b. Alternatively, if the healthcare transaction servers 102a-102c determines that the request does not satisfy the predetermined requirements (“NO” at block 216), the healthcare transaction servers 102a-102c may deny the request (block 208). It will be understood that the healthcare transaction servers 102a-102c may deny the request if, during the comparison, the prescription does not match a list of approved prescriptions (e.g., Zyrtec® may have been prescribed, but only Allegra® is an approved allergy medication which the insurer has agreed to cover under the patient's insurance plan). In embodiments, the healthcare transaction servers 102a-102c may also transmit information to the pharmacy workstation 108a, 108b indicating one or more equivalent prescriptions (e.g., similar chemical compounds) which may be substituted and that would be covered under the agreement between the patient and the patient's insurance company. The pharmacy may then either accept or deny the substitution and transmit an indication of the acceptance or denial via the pharmacy workstation 108a, 108b to the healthcare transaction servers 102a-102c. Once received, the healthcare transaction servers 102a-102c may update the entry in the ledger, indicating that the prescription has either been fulfilled or not fulfilled.

For example, if the prescribed treatment is an oral agent, the pharmacy can automatically send the token to the patient. When the patient picks up the treatment from the pharmacy, the patient can present this token to confirm her identity and receipt of treatment at the pharmacy counter. Alternatively, the specialty pharmacy can also send the drug directly to the patient by overnight delivery, and, at the time of delivery, the delivery person can confirm that the patient has the correct token when she digitally signs for the medication.

Referring to FIG. 3, illustrated is a system diagram of system components which may be included in or associated with computing devices such as the healthcare transaction servers 102a-102c, the patient workstations 104a, 104b, the clinician workstations 106a, 106b, and the pharmacy workstations 108a, 108b, the system diagram illustrating a system designated generally 300. The system 300 may include a memory 302, a processor 304, network interfaces 306, a display 308, one or more input devices 310, and an output module 316.

The display 308 may be any suitable display device capable of projecting images thereon, such as a liquid crystal display (LCD), a light-emitting diode (LED) display, and the like. The display 308 is in electrical communication with and configured to receive signals from, processor 304. As the processor 304 transmits signals to the display 308, the signals are converted to images which are output by display 308. The display 308 may further include integrated speakers (not explicitly shown) embedded in a housing of display 308, the integrated speakers configured to output audible signals based on the received signals from processor 304. Additionally, the display 308 may be configured to receive touch or tactile input, and subsequently transmit sensor signals indicative of the tactile input to processor 304.

The memory 302 may include transitory-type media, e.g., RAM, and/or non-transitory computer-readable media, e.g., flash media or disk media, for storing data. The data stored in memory 302 may include instructions or applications 312 executable by processor 304. The applications 312 may, when executed by the processor 304, cause the display 308 to display a certain image or images such as a user interface 314, enabling interaction between the users and the system 300. The processor 304, which is in electrical communication with memory 302, is configured to execute instructions or computer programs which are stored in memory 302 to augment or otherwise manipulate data stored in the memory 302. In this regard, the processor 304 includes any suitable logic control circuit adapted to perform calculations and/or operate according to a set of instructions. The instructions executed by processor 304 may control operation of the healthcare transaction servers 102a-102c, the patient workstations 104a, 104b, the clinician workstations 106a, 106b, and the pharmacy workstations 108a, 108b, including initiating transmission of data or communication therebetween. Additionally, the memory 302 may include instructions which enable reception of input data from input devices 310 or display 308. It is contemplated that memory 302 may be stored remotely and accessed, such remote storage and retrieval of data referred to generally in the art as cloud computing.

### Examples

When a clinician seeks to prescribe a treatment, the HealthGates™ portal (e.g., the healthcare transaction servers 102a-102c) transmit signals to present questions, or “GATES™,” to the clinician about the appropriateness of the prescription. These questions are based on proprietary compendia by HealthGates™ and the latest medical research. In general, the steps may involve the following:

1) Confirming patient genotype, mutations present or absent;

2) Confirming patient pathology recurrence phenotype, disease treatment resistant or not;

3) Confirming patient suitability for generic therapy, suitable or not;

4) Confirming method of administration and consistency of treatment, oral/IV, daily/weekly; and/or

5) Confirming prior answers and unlock the rights to administer/prescribe a drug.

For instance, if the doctor seeks to proscribe a drug called Zejula, the HealthGates™ Industry Portal may facilitate the following interaction:

QUESTION: CAN I USE Zejula FOR MY PATIENT AS MAINTENANCE THERAPY FOR OVARIAN CANCER?

Name of Patient:

Date of Birth:

MRN #:

1) Does the adult patient carry the diagnosis of recurrent epithelial carcinoma of the ovary, or fallopian tube cancer, or primary peritoneal carcinomatosis? Yes or No?

If No, then Zejula is not FDA indicated for this usage. Please consider alternative therapies.

If, Yes, please proceed to question #2.

2) Does the patient need initial therapy for recurrent/resistant disease? Yes or No?

If the answer is “Yes”, Zejula is FDA approved only as maintenance therapy for patients who have achieved a partial or complete response to a penultimate platinum containing therapy.

Please redirect your question to the specific condition and needed therapy of the patient.

If the answer is “No” for a therapy for recurrent/resistant disease and a yes for maintenance therapy, please proceed to question #3.

3) Has the patient achieved a partial or complete response to the penultimate platinum containing therapy? Yes or No?

If “No”, Zejula is not indicated for this purpose and please redirect your choice of therapy.

If “Yes” for a complete or partial response, advance to question #4.

4) Has it been a maximum of 8 weeks since the latest platinum based therapy? Yes or No?

If No, Zejula is indicated to be started within 8 weeks of the most recent platinum treatment.

Zejula is not FDA approved to be started more than 8 weeks since most recent platinum based therapy. Please redirect your therapy choices. If “yes”, that it is a maximum of 8 weeks since the last platinum based therapy, please proceed to question #5

5) Is this for oral administration? Yes or No?

If “yes”, for oral administration, then proceed to question #6. If “No” for oral administration, please redirect your inquiry.

6) Is this for a once a day administration of medicines? Yes or No?

If “No”, please redirect your questions to other forms of therapy as maintenance for ovarian, fallopian tube, or primary carcinomatosis.

If yes, then you have passed the HealthGates™ Portal for authorization for the use of Zejula for this patient. You may now proceed to the Preferred Specialty Pharmacy section to order this drug for your patient.

Would you like to proceed to the Specialty Pharmacy Portal?

If yes, please click on the following link (Specialty Pharmacy Portal).

If “No”, not at this time. Please click on the following link for approved authorizations and revisit this site at your convenience. (Approved Authorizations).

Once the physician passes the “GATES™,” she is able to write the relevant prescription. The patient may then obtain the drug from any specialty pharmacy within the HealthGates™ network. Both the patient and the pharmacy can be confident that the prescription is covered by insurance because participating insurance companies will agree to cover treatments that pass the GATES™ of HealtGates™.

For example, the prescription is sent from the physician's ERC20-compliant wallet to the pharmacy's ERC20-compliant wallet by transmitting an 18 decimal ERC20 GATES™ token. Each wallet has a unique address that identifies its holder. And once a physician sends a GATES™ token, the token contains information related to the physician, her practice, and the drug itself. Each GATES™ transaction carries a timestamp and data that identifies the prescribed treatment and encrypted data to confirm that the prescribing physician is the wallet's owner.

For example, if a doctor approved a 90 capsule, 100 mg regime of Zejula for a patient to pick up at a specific pharmacy (e.g., a CVS or Walgreens at a specified location). After passing through the GATES™, a transaction is initiated from his wallet, whose unique address is a long string of characters, such as 0xa4d98c5a890b05a4fd0ac5b2c029e1457051f6a3. His wallet would send a GATES™ token, which contains information about the prescribed drug, the intended patient, and confirming the doctor's identity, to the unique wallet for the specified pharmacy. This transaction would be recorded on a blockchain (e.g., Ethereum) and provide the insurance company and specialty pharmacy confirmation of activity. The patient gets an alert in her portal that her drug has been approved and is ready for pickup or delivery. The pharmacy's wallet sends an identifying GATES™ token number to the patient, which she will provide as proof of pre-authorized prescription at the pharmacy or at point of delivery.

Persons skilled in the art will understand that the structures and methods specifically described herein and illustrated in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, it is envisioned that the elements and features illustrated or described in connection with one exemplary embodiment may be combined with the elements and features of another without departing from the scope of the present disclosure and that such modifications and variations are also intended to be included within the scope of the present disclosure. Indeed, any combination of any of the presently disclosed elements and features is within the scope of the present disclosure. Accordingly, the subject matter of the present disclosure is not to be limited by what has been particularly shown and described.

What is claimed is:

1. A method for validating healthcare transactions via a web-based platform, the method including:
obtaining, by a server, a preliminary diagnosis from a remote computing device;
comparing, by the server, the preliminary diagnosis to a predetermined diagnosis criteria;
determining, by the server, whether the preliminary diagnosis satisfies the predetermined diagnosis criteria;
authorizing payment, by the server, to a predetermined entity when the preliminary diagnosis is determined to satisfy the predetermined diagnosis;
generating a unique token, by the server, when payment is authorized;
recording a transaction, by the server, in a distributed ledger based on the unique token to establish a recorded transaction;
associating, by the server, the unique token with the recorded transaction;
obtaining, by the server, a fulfillment request from the remote computing device;
validating, by the server, the recorded transaction based on the unique token to establish a validation; and
transmitting, by the server, an indication to fulfill the fulfillment request based on the validation to the remote computing device.

2. The method of claim 1, wherein the transaction includes an authorization for the payment to the predetermined entity and the preliminary diagnosis.

3. The method of claim 1, wherein the preliminary diagnosis includes a preliminary diagnosis information.

4. The method of claim 1, wherein the preliminary diagnosis includes a prescription information.

5. The method of claim 4, wherein the prescription information includes at least one of a name of a prescribing physician, a nature of a physician's practice, a name prescribed prescription, a dosage of the name prescribed prescription, a unique transaction serial number, or a transaction timestamp.

6. The method of claim 4, wherein the unique token includes a data related to the preliminary diagnosis.

7. The method of claim 6, wherein the unique token further includes a unique identifier.

8. The method of claim 1, wherein the preliminary diagnosis includes at least one of diagnosis information, scan data captured by medical imaging systems, or sample test data.

9. The method of claim 1, wherein the method further includes:
obtaining, by the server, an indication that the fulfillment request has been fulfilled; and
displaying an alert, on a user device, that the fulfillment request has been fulfilled.

10. A system for validating healthcare transactions via a web-based platform, the system including:
a server comprising: a processor and a memory storing instructions which, when executed by the processor, cause the system to:
obtain a preliminary diagnosis from a remote computing device;
compare the preliminary diagnosis to a predetermined diagnosis criteria;
determine whether the preliminary diagnosis satisfies the predetermined diagnosis criteria;
authorize payment to a predetermined entity when the preliminary diagnosis is determined to satisfy the predetermined diagnosis;
generate a unique token when payment is authorized;
record a transaction in a distributed ledger based on the unique token to establish a recorded transaction;
associate the unique token with the recorded transaction;
obtain a fulfillment request from the remote computing device;
validate the recorded transaction based on the unique token to establish a validation; and
transmit an indication to fulfill the fulfillment request based on the validation to the remote computing device.

11. The system of claim 10, wherein the transaction includes an authorization for the payment to the predetermined entity and the preliminary diagnosis.

12. The system of claim 10, wherein the preliminary diagnosis includes a preliminary diagnosis information.

13. The system of claim 10, wherein the preliminary diagnosis includes a prescription information.

14. The system of claim 13, wherein the prescription information includes at least one of a name of a prescribing physician, a nature of a physician's practice, a name prescribed prescription, a dosage of the name prescribed prescription, a unique transaction serial number, or a transaction timestamp.

15. The system of claim 13, wherein the unique token includes a data related to the preliminary diagnosis.

16. The system of claim 15, wherein the unique token further includes a unique identifier.

17. The system of claim 10, wherein the preliminary diagnosis includes at least one of diagnosis information, scan data captured by medical imaging systems, or sample test data.

18. The system of claim 10, wherein the instructions when executed further cause the system to:
obtain an indication that the fulfillment request has been fulfilled; and
display an alert, on a user device, that the fulfillment request has been fulfilled.

19. The system of claim 10, wherein the instructions when executed further cause the system to transmit the unique token to a user device.

20. A non-transitory computer-readable medium storing a program for validating healthcare transactions via a web-based platform, the program including instructions which, when executed, cause a server to:
obtain a preliminary diagnosis from a remote computing device;
compare the preliminary diagnosis to a predetermined diagnosis criteria;
determine whether the preliminary diagnosis satisfies the predetermined diagnosis criteria;
authorize payment to a predetermined entity when the preliminary diagnosis is determined to satisfy the predetermined diagnosis;
generate a unique token when payment is authorized;
record a transaction in a distributed ledger based on the unique token to establish a recorded transaction;
associate the unique token with the recorded transaction;
obtain a fulfillment request from the remote computing device;
validate the recorded transaction based on the unique token to establish a validation; and
transmit an indication to fulfill the fulfillment request based on the validation to the remote computing device.
